(19) United States
(12) Patent Application Publication
(10) Pub. No.: US 2020/0085409 A1
(43) Pub. Date: Mar. 19, 2020

(54) ULTRASOUND FOCUSING UTILIZING A 3D-PRINTED SKULL REPLICA

(71) Applicants: JAVIER GRINFELD, Tel Aviv-Yafo (IL); YOAV LEVY, Hinanit (IL)

(72) Inventors: JAVIER GRINFELD, Tel Aviv-Yafo (IL); YOAV LEVY, Hinanit (IL)

(21) Appl. No.: 16/132,630

(22) Filed: Sep. 17, 2018

(51) Int. Cl. A61B 8/00; A61N 7/00; G01S 15/89

(52) U.S. Cl. CPC A61B 8/4488; A61N 7/00; A61B 8/0808; A61N 2007/0004; A61N 2007/0056; G01S 15/8915

(57) ABSTRACT

Various approaches to transmitting an ultrasound beam include creating a 3D tissue replica representing tissue intervening between the ultrasound transducer and a target anatomic region; transmitting a ultrasound beam to the target region; measuring the ultrasound beam traversing the 3D tissue replica and arriving at the target region; and based at least in part on the measured first ultrasound beam, estimating a parameter value associated with one or more of the transducer elements for improving ultrasound beam shaping.

### FIELD OF THE INVENTION

The present invention relates, generally, to systems and methods for ultrasound focusing and, more particularly, to improved focusing using a three-dimensional (3D) printed skull replica.

### BACKGROUND

Focused ultrasound (i.e., acoustic waves having a frequency greater than about 20 kiloHertz) can be used to image or therapeutically treat a patient's internal body tissues. For example, ultrasound waves may be used in applications involving ablation of tumors, thereby eliminating the need for invasive surgery, targeted drug delivery, control of the blood-brain barrier, lysing of clots, and other surgical procedures. During tumor ablation, a piezoceramic transducer is placed externally to the patient, but in close proximity to the tissue to be ablated (i.e., the target). The transducer converts an electronic drive signal into mechanical vibrations, resulting in the emission of acoustic waves. The transducer may be geometrically shaped and positioned along with other such transducers so that the ultrasound energy they emit collectively forms a focused beam at a “focal zone” corresponding to (or within) the target tissue region. Alternatively or additionally, a single transducer may be formed of a plurality of individually driven transducer elements whose phases can each be controlled independently. Such a “phased-array” transducer facilitates steering the focal zone to different locations by adjusting the relative phases among the transducers. As used herein, the term “element” means either an individual transducer in an array or an independently drivable portion of a single transducer. Magnetic resonance imaging (MRI) may be used to visualize the patient and target, and thereby to guide the ultrasound beam.

The noninvasive nature of ultrasound surgery is particularly appealing for the treatment of brain tumors. However, treatment challenges arising from the anatomy of the human skull have limited the clinical realization of ultrasound therapy. Impediments to transcranial ultrasound procedures include strong attenuation and the distortions caused by irregularities in the skull's shape, density, and sound speed, which contribute toward destroying the ultrasound focus and/or decreasing the ability to spatially register diagnostic image information.

To overcome these difficulties, one conventional approach measures phase shifts resulting from travel of an ultrasound beam through the skull and subsequently adjusts ultrasound parameters to account for the aberrations caused at least in part by the skull. For example, a minimally invasive approach uses receiving probes designed for catheter insertion into the brain to measure the amplitude and phase distortion caused by the skull. Catheter insertions, however, still require surgery, which can be painful and can create a risk of infection.

An alternative, completely noninvasive approach uses X-ray computed tomography (CT) images, rather than receiving probes, to predict the wave distortion caused by the skull. In practice, however, computations of the relative phases alone may too be imprecise to enable high-quality focusing. For example, when ultrasound is focused into the brain to treat a tumor, the skull in the acoustic path may cause aberrations that are not readily ascertainable. As a result, the peak pressure of the focus generated using the image-based prediction approach may be only 80-85% of the peak pressure generated using ultrasound corrections made based on the probe measurements. Accordingly, there is a need for reliable approaches to correct for beam aberrations resulting from the skull during an ultrasound procedure and thereby allow for high-quality focusing at the target region.

### SUMMARY

The present invention provides systems and methods for focusing ultrasound beams that traverse tissue (such as a human skull) having an irregular structure, shape, density, and/or thickness onto a target region with a high-quality focus. For ease of reference, the following description only refers to an ultrasound treatment procedure; it should be understood, however, that the same approaches generally apply as well to an ultrasound imaging procedure. In addition, although the description herein refers to ultrasound beams traversing a human skull, the approach described in connection with various embodiments may be applied to determine beam aberrations resulting from any part of the human body, such as ribs, thereby allowing the parameter values characterizing an acoustic beam (e.g., phase shifts and/or amplitudes) to be adjusted to compensate for the aberrations.

In various embodiments, prior to ultrasound treatment, information characterizing the patient's skull, such as its anatomic characteristics (e.g., type, property, structure, thickness, density, etc.) and/or material characteristics (e.g., energy absorption of the tissue at the employed frequency or the speed of sound), is first acquired using, for example, an imaging device. Based on the obtained skull information, a patient-specific 3D skull replica may be created. For example, a 3D printing technique may be employed to create the skull replica using a material that has properties (e.g., the speed of sound) similar to that of the human skull. The 3D skull replica may then be situated in an environment similar to that used to treat the patient; a detector device (e.g., a hydrophone) may be deployed within the printed skull at the target region to measure acoustic signals that are transmitted from each of the ultrasound transducer elements during a simulated treatment sequence. By analyzing the measured signals, corrections to ultrasound parameters (e.g., amplitudes and/or phase shifts) associated with each transducer element may be determined. During treatment, the ultrasound transducer elements may be activated in accordance with the corrected ultrasound parameters so as to compensate for beam aberrations caused by the skull; this may thereby generate a high-quality focus at the target region and/or improve ultrasound beam shaping. In some embodiments, the area of the focal zone may be minimized to increase the peak acoustic intensity at the target region. In addition, the ultrasound beams transmitted from the transducer elements may be shaped such that the areas occupied thereby are minimized; this may avoid or minimize exposure of the non-target tissue to the therapeutic energy.

Because the material properties (e.g., stiffness and/or density) of the 3D skull replica may be different from those of the human skull, the ultrasound parameter corrections estimated using measurements of acoustic signals traversing the skull replica may need to be adjusted. In one embodiment, adjustment is based on measurements of ultrasound waves/pulses travelling through an ex-vivo skull. For example, a 3D skull replica that represents the ex-vivo skull may first be created as described above. The ultrasound waves/pulses traversing the 3D skull replica may then be detected using the detector device. The same measurement procedure may be similarly performed on the ex-vivo skull. Subsequently, signal measurements using the 3D skull replica may be compared against signal measurements using the ex-vivo skull. Based on the comparison, a proportionality mapping or an operator for adjusting the estimated ultrasound parameter corrections to account for the material difference between the skull replica and the human skull can be computed. This mapping/operator may then be generally applied to ultrasound parameter corrections estimated for other human skulls.

Alternatively, adjustment of the ultrasound parameter corrections may be performed using a “live” skull. For example, similar to the approaches described above, the ultrasound parameter corrections may be estimated using a 3D skull replica that represents the skull of a patient receiving the ultrasound treatment. During treatment or using retrospective study of the patient, corrections of the ultrasound parameters for achieving a desired focusing property at the target can be determined. Again, by comparing the corrections in the “live” case to the corrections estimated using the skull replica, the proportionality mapping/operator can be determined to account for the material difference between the patient's skull and the 3D skull replica.

Additionally or alternatively, a physical model may be implemented to adjust the estimated ultrasound parameter corrections based on measurements performed using the 3D skull replica. For example, the physical model may predict the beam path from each of the transducer elements to the target location based on information about the geometry of the transducer element and its location and orientation relative to the target; this information, in one implementation, is acquired using an imager. In addition, the physical model may include the anatomic/material characteristics of the patient's skull along the beam path from each transducer element to the target for predicting the aberrations resulting therefrom. The predicted aberrations may then be compared against the wave distortions measured using the 3D skull replica, and based thereon, the proportionality mapping/operator can be determined to update the ultrasound parameter corrections.

In some embodiments, the physical model further predicts the beam aberrations resulting from the printed 3D skull replica based on the anatomic/material properties thereof. The model-predicted aberrations may then be compared to the measured aberrations using the detector device. Again, based on the comparison, ultrasound parameter corrections estimated using the 3D skull replica may be adjusted.

During ultrasound treatment, the transducer elements may be activated in accordance with the estimated parameter corrections. Because the corrections are estimated using the patient-specific 3D skull replica, beam aberrations caused by the actual patient's skull may be accurately compensated for; consequently, a high-quality focus may be generated at the target. In some embodiments, the treatment effect resulting from the focus at the target is assessed during treatment. For example, a temporary local displacement of the target tissue resulting from acoustic radiation pressure and/or a temperature increase at the target region resulting from absorption of acoustic energy may be measured. The measured value may then be compared against a target objective. If the measured treatment effect slightly deviates from the target objective (e.g., within 10% or, in some embodiments, within 5%), the amplitudes and/or phase shifts of the transducer elements may be finely tuned (e.g., changed by less than 5%, or in some embodiments, changed by less than 1%) until the target objective is achieved. The tuned amplitudes and/or phase shifts may also be utilized to update the estimated parameter corrections. If, however, the measured temperature/tissue displacement differs significantly from the target objective (e.g., larger than 10% or, in some embodiments, 5%), transducer elements corresponding to large beam aberrations may be deactivated, or in some embodiments, the ultrasound frequency and/or the orientations and/or locations of the transducer elements related to the skull may be adjusted to reduce the aberrations therefrom.

Accordingly, the present invention advantageously utilizes a 3D skull replica representing a patient's skull to estimate ultrasound beam aberrations resulting therefrom. The ultrasound parameter values may then be corrected to compensate for the aberrations, thereby generating a high-quality focus at the target region and/or improving ultrasound beam shaping during treatment. In addition, because the corrections of the parameter values are estimated prior to treatment, they can be quickly looked up and utilized for ultrasound activation without prolonging the treatment procedure.

Accordingly, in one aspect, the invention pertains to a system for transmitting an acoustic beam including an ultrasound transducer having multiple transducer elements; a three-dimensional (3D) printed tissue replica representing tissue intervening between the ultrasound transducer and a target anatomic region; and a controller. In various embodiments, the controller is configured to (a) transmit the first ultrasound beam to the target region; (b) measure the first ultrasound beam traversing the 3D tissue replica and arriving at the target region; and (c) based at least in part on the measured first ultrasound beam, estimate a parameter value (e.g., a frequency, an amplitude, a time delay and/or a phase shift of the first ultrasound beam) associated with one or more of the transducer elements for improving ultrasound beam shaping. In one implementation, the system further includes a detector device for measuring the first ultrasound waves at the target region.

The system may include an imaging device for acquiring images of the intervening tissue; the 3D tissue replica may be generated based at least in part on the acquired images. In addition, the system may include a 3D printer for generating the 3D tissue replica. In some embodiments, the system further includes memory for storing the estimated parameter value associated with the transducer element(s). The controller may then be further configured to retrieve the stored parameter value and cause the transducer element(s) to generate the second ultrasound beam based at least in part on the stored parameter value. In one embodiment, the controller is further configured to sequentially cause at least some of the transducer elements to transmit ultrasound beams to the target region; sequentially measure the transmitted ultrasound beams traversing the 3D tissue replica and arriving at the target region; based at least in part on the measured ultrasound beams, estimate multiple parameter values associated with the transducer elements; and store the estimated parameter values in the memory.

In addition, the controller may be further configured to adjust the estimated parameter value using a physical model. For example, the controller may be further configured to use the physical model to predict a beam path from the transducer element(s) to the target region based at least in part on the geometry of the transducer element(s) and its(their) location(s) and orientation(s) relative to the target region. In one embodiment, the controller is further configured to use the physical model to predict the parameter value associated with the transducer element(s) based at least in part on tissue characteristics of the intervening tissue along the beam path; and adjust the estimated parameter value based at least in part on the prediction. Additionally or alternatively, the controller may be configured to use the physical model to predict the parameter value associate with the transducer element(s) based at least in part on the material property of the 3D tissue replica; and adjust the estimated parameter value based at least in part on the prediction. In various embodiments, the controller is further configured to cause the second ultrasound beam to be transmitted to the target region; measure the second ultrasound beam arriving at the target region after penetrating through the intervening tissue; based at least in part on the measured second ultrasound beam, estimate the second parameter value associated with the transducer element(s); and adjust the estimated parameter value based at least in part on the estimated second parameter value.

In addition, at least some of the transducer elements may be activated to generate an ultrasound focus at the target region; the system may further include a measurement system for monitoring treatment effects (e.g., a temperature increase and/or a tissue displacement) of the target region resulting from the ultrasound focus. The controller may be further configured to adjust the estimated parameter value based at least in part on the monitored treatment effects. In one embodiment, the controller is configured to adjust the second parameter value (e.g., a frequency, a location and/or an orientation) associated with the transducer element(s) based at least in part on the monitored treatment effects. Generally, the second parameter value is different from the estimated parameter value.

In some embodiments, the 3D tissue replica and the ultrasound transducer have a spatial configuration; the controller is further configured to determine, based at least in part on the measured first ultrasound beam, an optimal spatial configuration of the 3D tissue replica and the ultrasound transducer. The spatial configuration may include a relative orientation and/or location of the 3D tissue replica with respect to the ultrasound transducer. In addition, the controller may be further configured to vary the spatial configuration of the 3D tissue replica and the ultrasound transducer; repeat steps (a)-(c); and based at least in part on the measured ultrasound beams, determine the optimal spatial configuration. In one implementation, the controller is further configured to determine the optimal spatial configuration using a physical model in addition to the measured first ultrasound beam.

In another aspect, the invention relates to a method of transmitting an acoustic beam from an ultrasound transducer having multiple transducer elements. In various embodiments, the method includes creating a 3D tissue replica representing tissue intervening between the ultrasound transducer and a target anatomic region; transmitting the first ultrasound beam to the target region; measuring the first ultrasound beam traversing the 3D tissue replica and arriving at the target region; and based at least in part on the measured first ultrasound beam, estimating a parameter value (e.g., a frequency, an amplitude, a time delay and/or a phase shift of the first ultrasound beam) associated with one or more of the transducer elements for improving ultrasound beam shaping. In one implementation, the 3D tissue replica is created using 3D printing. In addition, the method may further include acquiring images of the intervening tissue; the 3D tissue replica is generated based at least in part on the acquired images.

In some embodiments, the method further includes storing the estimated parameter value associated with the transducer element(s). In addition, the method may further include retrieving the stored parameter value and causing the transducer element(s) to generate the second ultrasound beam based at least in part on the stored parameter value. In one embodiment, the method further includes sequentially causing at least some of the transducer elements to transmit beams to the target region; sequentially measuring the transmitted ultrasound beams traversing the 3D tissue replica and arriving at the target region; based at least in part on the measured ultrasound beams, estimating parameter values associated with the transducer element(s); and storing the estimated parameter values in the memory.

In addition, the method may further include adjusting the estimated parameter value using a physical model. For example, the method may include using the physical model to predict a beam path from the transducer element(s) to the target region based at least in part on the geometry of the transducer element(s) and its(their) location(s) and orientation(s) relative to the target region. In one embodiment, the method includes using the physical model to predict the parameter value associated with the transducer element(s) based at least in part on tissue characteristics of the intervening tissue along the beam path; and adjusting the estimated parameter value based at least in part on the prediction. Additionally or alternatively, the method may further include using the physical model to predict the parameter value associate with the transducer element(s) based at least in part on the material property of the 3D tissue replica; and adjusting the estimated parameter value based at least in part on the prediction. In various embodiments, the method further includes transmitting the second ultrasound beam to the target region; measuring the second ultrasound beam arriving at the target region after penetrating through the intervening tissue; based at least in part on the measured second ultrasound beam, estimating the second parameter value associated with the transducer element(s); and adjusting the estimated parameter value based at least in part on the estimated second parameter value.

In addition, the method may further include activating at least some of the transducer elements to generate an ultrasound focus at the target region and monitoring treatment effects (e.g., a temperature increase and/or a tissue displacement) of the target region resulting from the ultrasound focus. The method may further include adjusting the estimated parameter value based at least in part on the monitored treatment effects. In one embodiment, the method further includes adjusting the second parameter value (e.g., a frequency, a location and/or an orientation) associated with the transducer element(s) based at least in part on the monitored treatment effects. Generally, the second parameter value is different from the estimated parameter value.

In some embodiments, the 3D tissue replica and the ultrasound transducer have a spatial configuration; the method then further includes determining, based at least in part on the measured first ultrasound beam, an optimal spatial configuration of the 3D tissue replica and the ultrasound transducer. The spatial configuration may include a relative orientation and/or location of the 3D tissue replica with respect to the ultrasound transducer. In addition, the method may further include varying the spatial configuration of the 3D tissue replica and the ultrasound transducer; repeating steps (a)-(c); and based at least in part on the measured ultrasound beams, determining the optimal spatial configuration. In one implementation, the method further includes determining the optimal spatial configuration using a physical model in addition to the measured first ultrasound beam.

As used herein, the term “replica” means a structure that is substantially similar in its exterior three-dimensional shape to the anatomic structure that it models, e.g., a particular patient's skull. However, the replica may omit certain structural details that do not significantly affect the shape of the exterior surface and/or whose omission is not clinically relevant to the aberration of ultrasound waves/pulses traversing the replica. Such replicas are considered “substantially similar in shape and/or structure.”. More generally, the term “substantially” or “approximately” means ±10%, and in some embodiments, ±5% of the peak intensity. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

### BRIEF DESCRIPTION OF THE DRAWINGS

In the drawings, like reference characters generally refer to the same parts throughout the different views. Also, the drawings are not necessarily to scale, with an emphasis instead generally being placed upon illustrating the principles of the invention. In the following description, various embodiments of the present invention are described with reference to the following drawings, in which:

FIG. 1 illustrates a focused ultrasound system in accordance with various embodiments;

FIG. 2 schematically illustrates tissue layers of a human skull;

FIG. 3 illustrates a 3D skull replica in a focused ultrasound system in accordance with various embodiments;

FIG. 4A is a flow chart illustrating an approach for determining ultrasound parameter values to compensate for beam aberrations resulting from intervening tissue (e.g., the skull) located between the transducer and the target in accordance with various embodiments; and

FIG. 4B is a flow chart illustrating an exemplary approach for optimizing focusing properties at the target region during an ultrasound procedure in accordance with various embodiments.

### DETAILED DESCRIPTION

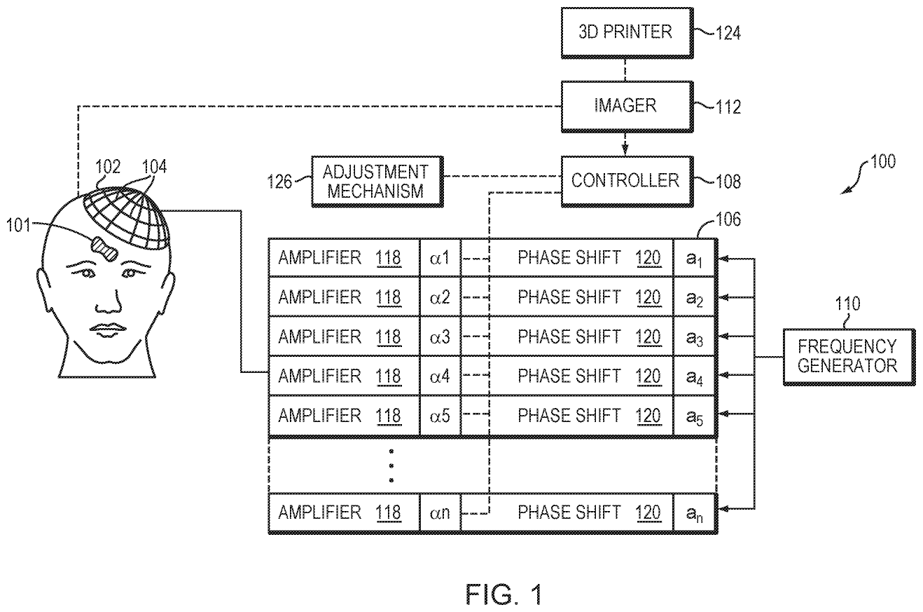

FIG. 1 illustrates an exemplary ultrasound system 100 for focusing ultrasound onto a target region 101 through the skull. One of ordinary skill in the art, however, will understand that the ultrasound system 100 described herein may be applied to any part of the human body. In various embodiments, the system 100 includes a phased array 102 of transducer elements 104, a beamformer 106 driving the phased array 102, a controller 108 in communication with the beamformer 106, and a frequency generator 110 providing an input electronic signal to the beamformer 106.

The array 102 may have a curved (e.g., spherical or parabolic) shape suitable for placing it on the surface of the skull or a body part other than the skull, or may include one or more planar or otherwise shaped sections. Its dimensions may vary, depending on the application, between millimeters and tens of centimeters. The transducer elements 104 of the array 102 may be piezoelectric ceramic elements, and may be mounted in silicone rubber or any other material suitable for damping the mechanical coupling between the elements 104. Piezo-composite materials, or generally any materials capable of converting electrical energy to acoustic energy, may also be used. To assure maximum power transfer to the transducer elements 104, the elements 104 may be configured for electrical resonance at 50Ω, matching input connector impedance.

The transducer array 102 is coupled to the beamformer 106, which drives the individual transducer elements 104 so that they collectively produce a focused ultrasonic beam or field. For n transducer elements, the beamformer 106 may contain n driver circuits, each circuit including or consisting of an amplifier 118 and a phase delay circuit 120; drive circuit drives one of the transducer elements 104. The beamformer 106 receives a radio frequency (RF) input signal, typically in the range from 0.1 MHz to 10 MHz, from the frequency generator 110, which may, for example, be a Model DS345 generator available from Stanford Research Systems. The input signal may be split into n channels for the n amplifiers 118 and delay circuits 120 of the beamformer 106. In some embodiments, the frequency generator 110 is integrated with the beamformer 106. The radio frequency generator 110 and the beamformer 106 are configured to drive the individual transducer elements 104 of the transducer array 102 at the same frequency, but at different phases and/or different amplitudes, such that the transducer elements 104 collectively form a “phased array.”

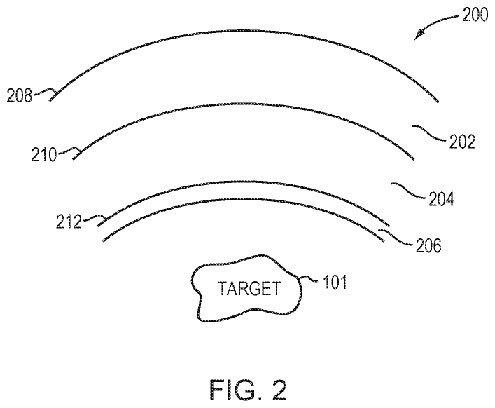

The acoustic waves/pulses transmitted from the transducer elements 104 form an acoustic energy beam. Typically, the transducer elements are driven so that the waves/pulses converge at a focal zone in the targeted tissue 101. Within the focal zone, the wave energy of the beam is (at least partially) absorbed by the tissue, thereby generating heat and raising the temperature of the tissue to a point where the cells are denatured and/or ablated. To effectively treat the target tissue, the acoustic energy beam must be precisely focused to the target location 101 to avoid damage to healthy tissue surrounding the target region. Referring to FIG. 2, a typical human skull 200, however, is inhomogeneous and has multiple tissue layers, including an external layer 202, a bone marrow layer 204, and an internal layer or cortex 206; each layer of the skull 202 may be highly irregular in shape, thickness and density, and unique to a patient. As a result, when the ultrasound waves/pulses emitted from the system 100 encounter the skull 200, beam scattering, absorption, reflection, and/or refraction may occur due to tissue inhomogeneity; this may result in beam aberrations, which may distort the focus and reduce the intensity, thus affecting treatment efficiency. Accordingly, it is desired to adjust parameters (e.g., the phase shifts a₁-aₙ and/or amplification or attenuation factors α₁-αₙ) of the drive signals associated with the transducer elements so as to compensate for the acoustic aberrations and thereby improve focusing properties at the target region 101.

Generally, the amplification factors and phase shifts may be computed using the controller 108, which may provide the computational functions through software, hardware, firmware, hardwiring, or any combination thereof. For example, the controller 108 may utilize a general-purpose or special-purpose digital data processor programmed with software in a conventional manner, and without undue experimentation, to determine the parameters (e.g., frequencies, phase shifts and/or amplification factors) of the transducer elements 104. The controller 108 may determine the parameters based on information about the characteristics (e.g., structure, thickness, density, etc.) of the skull and their effects on propagation of acoustic energy. Referring again to FIG. 1, in one embodiment, such information is obtained from an imager 112, such as a magnetic resonance imaging (MRI) device, a computer tomography (CT) device, a positron emission tomography (PET) device, a single-photon emission computed tomography (SPECT) device, or an ultrasonography device.

In some embodiments, the ultrasound parameter values that can be used to compensate for beam aberrations resulting from the skull may be determined based on acoustic measurements of ultrasound waves traversing the skull. For example, prior to and/or during the ultrasound procedure, a CT device may first acquire images of the patient's skull; the CT images may be 3D images or a set of two-dimensional (2D) images suitable for reconstructing a 3D image of the skull from which thicknesses and densities can be inferred (image-manipulation functionality may be implemented in the imager 112, in the controller 108, or in a separate device). Based on the reconstructed 3D image, a 3D skull replica having anatomical characteristics (such as the skull thickness, local bone densities and/ or directional or geometrical features including a normal relative to a surface region of the skull 200) of the skull 200 may be formed in any suitable manner. For example, a 3D printer 124 may be implemented to deposit a suitable material in a layer-by-layer manner onto a surface so as to build up the 3D replica (this process is often termed “additive manufacture” or “3D printing”); 3D printers are conventional and readily available. Generally, the material used for the printed replica is selected such that the speed of sound therein is substantially similar to that in the skull 200. Because the skull layers 202-206 may have different densities and stiffness, their associated speeds of sound may be different. Thus, multiple materials may be employed to form the 3D skull replica. Polymeric materials commonly used in 3D printing, and which may be suitably employed herein, include, without limitation, ABS plastic, polyactic acid (PLA), polyamide (NYLON), glass-filled polyamide, stereolithography materials including epoxy resins, and polycarbonate.

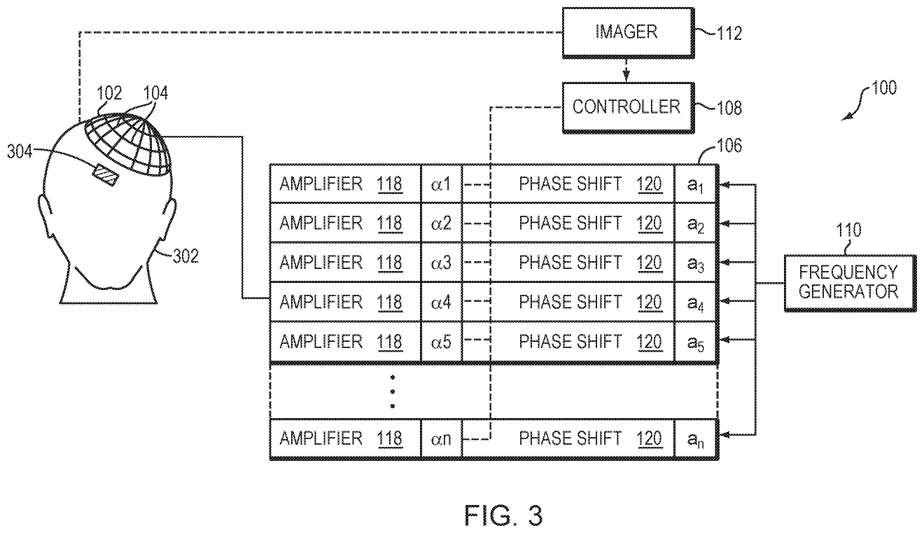

Referring to FIG. 3, once printed, the 3D skull replica 302 may be situated in an environment similar to that used to treat the patient during the ultrasound procedure (e.g., over a water bath containing the ultrasound transducer 102 or inside an MRI apparatus). In addition, a detector device (e.g., a hydrophone) 304 may be placed at the target location 101 for measuring acoustic signals transmitted from the transducer 102, penetrating through the printed 3D skull replica 302 and finally arriving at the target location 101. In one embodiment, the transducer elements 104 are sequentially activated (one at a time) to transmit a short pulse (e.g., 20 cycles) to the target region 101; the hydrophone 304 then detects the pulse arriving at the target region 101 and subsequently transmits the detected signal to the controller 108. In some embodiments, the controller 108 analyzes the received signal to determine the amplitude and/or phase shift associated therewith. In addition, based on the relative orientation and/or location of the target location 101 with respect to the activated transducer element 104, the controller 108 may compute the expected amplitude and/or phase shift associated with the ultrasound pulse arriving at the target 101 in the absence of the skull 200 (and the media located between the skull 200 and the transducer 102). By comparing the measured amplitude and/or phase shift against the expected amplitude and/or phase shift in the absence of the skull, ultrasound parameter corrections associated with the activated element 104 for compensating for the beam aberration resulting from the skull can be determined. For example, assuming that the expected phase shift in the absence of the skull is φₑ and the measured phase shift is φₘ, the aberration Δφ caused by the skull can be computed as Δφ=φₘ−φ_{c}. Therefore, by correcting the phase shift of the ultrasound pulses emitted from the activated transducer element to account for the aberration Δφ, a focus with desired properties may be generated at the target region 101. This procedure can be repeated for all (or at least a portion) of the transducer elements 104. It should be noted that different imaging systems may be involved in determining the relative orientation and/or location of the target 101 with respect to the transducer elements 104. For example, the orientations and locations of the transducer elements 104 may be obtained using, e.g., a time-of-flight approach in the ultrasound system, whereas the spatial characteristics of the target region 101 may be acquired using MRI. As a consequence, it may be necessary to register coordinate systems in different imaging modalities prior to computing the expected amplitude and/or phase shift associated with each transducer element. Exemplary registration approaches are provided, for example, in U.S. Pat. No. 9,934,570, the entire disclosures of which are hereby incorporated by reference.

In another embodiment, the controller 108 further computes the “time of flight” (TOF) of the acoustic waves/pulses emitted by the transducer elements 104 and detected by the hydrophone 304 at the target 101. Based on the TOF, a time delay caused by the skull can be determined. Accordingly, the ultrasound parameter value associated with each element 104 can be adjusted to compensate for its corresponding time delay. Additionally or alternatively, the controller 108 may adjust the amplitude (intensity) of each transducer element 104 based on the measured aberration Δφ. For example, when Δφ exceeds a predetermined threshold, the aberration resulting from the skull replica 302 may be sufficiently significant to cause overheating of the skull; thus, in one embodiment, the amplitude of the transducer element 104 associated therewith is reduced to avoid damage to non-target tissue. Alternatively, the controller 108 may deactivate the associated transducer element 104 to avoid damage. In addition, the focus of the acoustic beams traversing the skull replica 302 may be quantitatively assessed as further described below. Based on the assessment, the amplitudes of transducer elements may be adjusted for achieving a desired focusing property (e.g., maximize the peak acoustic power, generate a desired focus shape, etc.).

In various embodiments, the determined ultrasound parameter corrections (including the amplitudes, time delays and/or phase shifts) and/or the activation/deactivation pattern are stored along with their respective transducer elements in a database in memory accessible by the controller 108. In one implementation, the database stores the transducer elements and their corresponding phase corrections resulting from the skull in a look-up acoustic-correction table (ACT). The memory may include or consist essentially of one or more volatile or non-volatile storage devices, e.g., random-access memory (RAM) devices such as DRAM, SRAM, etc., read-only memory (ROM) devices, magnetic disks, optical disks, flash memory devices, and/or other solid-state memory devices. All or a portion of the memory may be located remotely from the ultrasound system 100 and/or the imager 112, e.g., as one or more storage devices connected to ultrasound system 100 and/or the imager 112 via a network (e.g., Ethernet, WiFi, a cellular telephone network, the Internet, or any local- or wide-area network or combination of networks capable of supporting data transfer and communication). As utilized herein, the term “storage” broadly connotes any form of digital storage, e.g., optical storage, magnetic storage, semiconductor storage, etc.

In one embodiment, the controller 108 may implement a physical model to predict treatment effects of the target region 101 using tissue characteristics (e.g., the energy absorption coefficient) thereof and the ACT file. Based on the predicted treatment effects, the controller 108 may determine patient suitability for ultrasound treatment. In various embodiments, the tissue characteristics of the target region are acquired using the imager 112. For example, based on the acquired images, a tissue model characterizing the material characteristics of the target region may be established. The tissue model may take the form of a 3D table of cells corresponding to the voxels representing the target tissue; the cells have attributes whose values represent characteristics of the tissue, such as the absorption coefficient, that are relevant to the energy absorption. The voxels are obtained tomographically by the imaging device and the type of tissue that each voxel represents can be determined automatically by conventional tissue-analysis software. Using the determined tissue types and a lookup table of tissue parameters (e.g., absorption coefficient by type of tissue), the cells of the tissue model may be populated. Further detail regarding creation of a tissue model that identifies the energy absorption coefficient, heat sensitivity and/or thermal energy tolerance of various tissues may be found in U.S. Patent Publication No. 2012/0029396, the entire disclosure of which is hereby incorporated by reference.

In some embodiments, the spatial coordinates of the 3D skull replica 302 with respect to the transducer elements 104 are adjusted to optimize the treatment effect. For example, due to structural inhomogeneity, different skull regions may have different transmission efficiencies. Accordingly, in one implementation, the relative orientation and/or location of the 3D skull replica 302 with respect to the transducer elements 104 is adjusted such that a majority of the ultrasound waves/pulses from the transducer elements 104 traverse the skull regions corresponding to sufficiently high transmission efficiency (e.g., above a predetermined threshold, such as 0.5, 0.8 or 0.9). Approaches to determining the transmission efficiencies associated with various skull regions are provided, for example, in U.S. patent application Ser. No. 15/708,214, the contents of which are incorporated herein by reference.

Referring again to FIG. 1, the system 100 may further include an adjustment mechanism (e.g., a motor, a gimbal, or other manipulator) 126 that is responsive to a communication from the controller 108 and permits mechanical adjustment of the orientation (e.g., an angle or a position) and/or translation (if desired) of the 3D skull replica 302 and/or transducer elements 104. For example, the adjustment mechanism 126 may physically rotate the 3D skull replica 302 around one or more axes thereof and/or move the 3D skull replica 302 with respect to the transducer to a different location. In some embodiments, the skull replica 302 and transducer 102 have a discrete number of predetermined spatial configurations relative to each other. The ultrasound parameter correction approaches described above may be performed in each spatial configuration. Based on the measured aberrations Δφ of the transducer elements 104, the controller 108 may determine the spatial configuration corresponding to an optimal treatment scenario (e.g., having a minimal total aberration of the ultrasound waves/pulses traversing the skull replica 302). This may advantageously reduce skull heating and/or optimize the focusing property at the target region 101.

It should be stressed that it may not be unnecessary to perform the ultrasound aberration measurements for each spatial configuration of the skull replica 302 and transducer 102. For example, after the ultrasound aberrations associated with a spatial configuration are measured, the physical model described above may predict the ultrasound aberrations associated with one or more other spatial configurations based on the measured aberrations and/or the tissue model characterizing the material characteristics of the skull and/or target region. Accordingly, following the initial measurement in a particular configuration, the optimal configuration corresponding to the minimal total aberration of acoustic beams traversing the 3D skull replica 302 may be determined based on the model. Alternatively, the optimal configuration may be determined based on the assessment of the focus at the target region 101. Once again, the determined optimal configuration of the skull replica 302 and the transducer array 102 and the associated ACT file may be stored in the database accessible by the controller 108.

In various embodiments, during the ultrasound procedure, the controller 108 may retrieve information stored in the look-up table and drive the transducer elements 104 based on their associated parameter corrections. Optionally, the controller 108 may adjust the spatial configuration of the patient's skull and the transducer array 102 based on the retrieved information. Because the printed 3D skull replica 302 is established based on the CT images of an individual patient's skull, parameter corrections determined based on measurements of the ultrasound pulses traversing the printed skull replica may accurately compensate for aberrations caused by the patient-specific skull, thereby advantageously allowing a high-quality focus to be properly located at the target 101 for individual patients as well as improving ultrasound beam shaping.

In some embodiments, the detector device 304 is mounted movably and rotatably on a conventional actuator or scanner, which may be driven by a component of controller 108 or by a separate mechanical controller. As a result, the detector device 304 may be moved easily in the printed skull replica 302 to facilitate acoustic signal measurements at multiple target regions as described above. Typically, one ultrasound ACT file is created for one target region; during the ultrasound procedure, the ACT file is retrieved based on the target to be treated.

Because the properties (e.g., stiffness and density) of the material utilized to print the 3D skull replica 302 may be different from that of the human skull, the speed of sound (and therefore beam aberrations) may also differ; as a result, the ultrasound parameter corrections in the ACT file may need to be adjusted to account for the material difference. In some embodiments, compensation may be achieved by simply multiplying all relevant pixel attributes by a proportionality constant. In other embodiments, however, the required adjustment is unknown, or varies across the skull, or is nonlinear; in such cases, the relationship may be modeled empirically based on measurements performed using an ex-vivo skull. For example, the imager (e.g., CT device) 112 may first acquire images of the ex-vivo skull; based on the acquired images, a 3D skull replica representing the ex-vivo skull can be printed as described above. The printed skull replica may then be situated in the location and environment which preferably (but not necessarily) are the same as the location and environment in which the patient's skull will be situated during the ultrasound procedure. Again, the detector device 304 is employed to measure the acoustic signals from each of the transducer elements 104 traversing the printed skull replica and arriving at the target location 101. In one embodiment, this procedure is repeated by replacing the printed skull replica 302 with the ex-vivo skull. Subsequently, the measured acoustic signals using the printed skull replica 302 that represents the ex-vivo skull can be compared against the measured acoustic signal using the real ex-vivo skull. Based on the comparison, a proportionality mapping or operator may be computed to adjust the estimated ultrasound parameter corrections using the printed skull replica so as to account for the material difference between the skull replica and the ex-vivo skull. The ultrasound parameter corrections stored in the ACT file may then be updated accordingly. In addition, the mapping/operator (which may include a linear or a non-linear function) may be stored in the database as well. Because the mapping/operator represents the difference in material properties between the printed skull replica and the human skull, it can be generally applied to correct future ACT files that are created using the same material for 3D skull replica.

In some embodiments, corrections to the ACT file are performed using a “live” skull. For example, similar to the approaches described above, the ACT file of a patient who previously experienced the ultrasound procedure may be created prior to or after treatment. During treatment, corrections of the ultrasound parameters for achieving a desired focusing property at the target 101 can be empirically determined. Thus, the corrections may be facilitated by employing an acoustic reflector substantially close to the target region 101 such that ultrasound waves/pulses transmitted from all (or at least some) transducer elements 104 are reflected by the reflector. By analyzing the reflected signals, the controller 108 may obtain information, such as the amplitudes and/or phases, associated therewith for determining the corrections of the ultrasound parameter values for achieving the desired focusing property. In one embodiment, the acoustic reflector consists essentially of microbubbles generated by the ultrasound waves/pulses and/or introduced parenterally by an administration system. Approaches to generating the microbubbles and/or introducing them into the target region 101 are provided, for example, in U.S. patent application Ser. Nos. 62/366,200, 62/597,071, 15/708,214, 5/837,392 and 62/597,073, the contents of which are incorporated herein by reference. In addition, the transducer elements 104 may possess both transmit and detect capabilities; thus, the reflected signals from the acoustic reflector can be detected by the transducer elements 104. Approaches to configuring the transducer elements for detecting the reflected signals are provided, for example, in the U.S. Patent Application No. 62/861,282, the contents of which are incorporated herein by reference. Alternatively, the ultrasound parameter corrections may be determined based on retrospective study after treatment. In various embodiments, the corrections in the “live” case can be compared to the corrections stored in the ACT file, and again, based thereon, the proportionality mapping therebetween can be estimated.

Additionally or alternatively, the controller 108 may implement a physical model to adjust the ultrasound parameter corrections stored in the ACT file. For example, the physical model may predict the beam path from each of the transducer elements 104 to the target location 101 using information about the geometry of the transducer element 104 and its location and orientation relative to the target 101. This information, in one implementation, is acquired using the imager 112. For example, an MRI apparatus may be utilized to acquire images of the target. The MRI imaging system may then be registered to the ultrasound system in order to determine the relative locations between the transducer elements 104 and target 101. Approaches to registering images acquired using two or more imaging systems are provided, for example, in U.S. Pat. No. 9,934,570, the entire disclosure of which is hereby incorporated by reference. In addition, the physical model may take into account transducer output errors resulting from, for example, transducer elements 104 moving or shifting from their expected location during manufacturing, use and repair and/or as a result of the elements 104 being deformed by heat. Additional information concerning the approach of determining the transducer output errors is provided in U.S. Pat. No. 7,535,794, the contents of which are incorporated herein by reference.

In some embodiments, the physical model further includes anatomic characteristics (e.g., the type, property, structure, thickness, density, etc.) and/or material characteristics (e.g., the energy absorption of the tissue at the employed frequency or the speed of sound) of the patient's skull along the beam path for predicting the aberrations resulting therefrom. For example, based on the anatomic/material properties, time delays of the ultrasound pulses/waves penetrating through the skull may be estimated; the time delays may then be converted to phase shifts that need to be compensated for. Again, the anatomic/material properties may be collected using the imager 112 (such as a CT device) and/or other suitable devices.

In some embodiments, the physical model further computes ultrasound parameter corrections required to compensate for the predicted aberrations. The model-prediction corrections may then be compared against the information in the ACT on an element-by-element basis. If the deviation therebetween for a particular element 104 is below a predetermined threshold, the parameter correction for that element stored in the ACT file may be adjusted. For example, the stored parameter correction may be adjusted to match the model-predicted value. Alternatively, an average of the stored correction and model-predicted correction may be utilized as the updated correction stored in the ACT file. If, however, the deviation exceeds the predetermined threshold, the measurement accuracy of the acoustic signals at the target 101 may have to be improved (e.g., by increasing the signal-to-noise ratio) and/or the physical model may have to be adjusted (e.g., using additional imaging data).

In other embodiments, the physical model predicts the beam aberrations resulting from the printed 3D skull replica 302 based on the anatomic properties (e.g., the structure, thickness, density, etc.) and/or material properties (e.g., the speed of sound) thereof. The controller 108 may then compare the model-predicted value to the measured value using the detector device 304. Again, based on the comparison, ultrasound parameter corrections stored in the ACT may be adjusted using the approaches described above.

During the ultrasound procedure, the controller 108 may retrieve the stored ACT file and activate the transducer elements based thereon. Because the ACT file is patient-specific, beam aberrations caused by the individual patient's skull may be accurately compensated for; as a result, a high-quality focus may be generated at the target 101. In some embodiments, the focus at the target 101 is quantitatively assessed for evaluating the parameter corrections in the ACT file, adjusting the treatment protocol and/or determining whether the patient is suitable for the ultrasound procedure. Various techniques can be used to assess the focus—directly, or indirectly via a related physical quantity. One approach is to measure the temporary local displacement of the target tissue resulting from acoustic radiation pressure, which is highest at the focus (where the ultrasound waves converge and highest intensity is achieved). The ultrasound pressure creates a displacement field that directly reflects the acoustic field. The displacement field can be visualized, using a technique such as MR-ARFI, by applying transient-motion or displacement-sensitizing magnetic field gradients to the imaging region by gradient coils, which are part of standard MRI apparatus. When the ultrasound pulse is applied in the presence of such gradients, the resulting displacement is directly encoded into the phase of the MR response signal. For example, the gradient coils and transducer may be configured such that the ultrasound pulse pushes material near the focus towards regions of the magnetic field with higher field strengths. In response to the resulting change in the magnetic field, the phase of the MR response signal changes proportionally, thereby encoding in the signal the displacement caused by the ultrasound radiation pressure. Further detail about MR-ARFI is provided in U.S. Pat. No. 8,932,237, the entire disclosure of which is hereby incorporated herein by reference.

Another quantity usefully related to assessing the focus is the temperature at the target and/or non-target regions, which increases proportionally to the amount of acoustic energy delivered thereto. Thermometry methods may be based, e.g., on MRI, in conjunction with suitable image-processing software. Among various methods available for MR thermometry, the proton resonance frequency (PRF) shift method is often the method of choice due to its excellent linearity with respect to temperature change, near-independence from tissue type, and temperature map acquisition with high spatial and temporal resolution. The PRF shift method exploits the phenomenon that the MR resonance frequency of protons in water molecules changes linearly with temperature. Since the frequency change with temperature is small, only −0.01 ppm/° C. for bulk water and approximately −0.0096 to −0.013 ppm/° C. in tissue, the PRF shift is typically detected with a phase-sensitive imaging method in which the imaging is performed twice: first to acquire a baseline PRF phase image prior to a temperature change and then to acquire a second phase image after the temperature change, thereby capturing a small phase change that is proportional to the change in temperature. A map of temperature changes may then be computed from the MR images by determining, on a pixel-by-pixel basis, phase differences between the baseline image and the treatment image, and converting the phase differences into temperature differences based on the PRF temperature dependence while taking into account imaging parameters such as the strength of the static magnetic field and echo time (TE) (e.g., of a gradient-recalled echo). Various alternative or advanced methods may be used to compensate for patient motion, magnetic-field drifts, and other factors that affect the accuracy of PRF-based temperature measurements; suitable methods known to those of skill in the art include, e.g., multibaseline and referenceless thermometry.

It should be noted that one of ordinary skill in the art will understand that approaches to assessing the focusing property at the target is not limited to measurements of the temperature and tissue displacement; any other parameter(s) suitable as an indicator for the focusing property at the target can be measured and are thus within the scope of present invention.

In various embodiments, the measured temperature and/or tissue displacement indicating the focusing property at the target is compared against the target objective. Based on the comparison, the ultrasound parameter corrections in the ACT file may be further adjusted. For example, if the measured temperature/tissue displacement slightly deviates from the target objective (e.g., within 10% or, in some embodiments, within 5%), the amplitudes and/or phase shifts of the transducer elements may be finely tuned until the target objective is achieved; the amplitudes and/or phase shifts may then be used to update the ultrasound parameter corrections in the ACT file. If, however, the measured temperature/tissue displacement differs significantly from the target objective (e.g., larger than 10% or, in some embodiments, 5%), transducer elements 104 corresponding to large beam aberrations may be deactivated during treatment to reduce distorting of the focus. Additionally or alternatively, the ultrasound frequency and/or the orientation and/or location of the transducer elements related to the skull may be adjusted to reduce the aberrations therefrom. In this situation, the ACT file may have to be updated by, for example, transmitting ultrasound waves/pulses having the adjusted frequency and/or transmitting ultrasound waves/pulses from the adjusted element locations/orientations through the skull, and measuring the resulting signals at the target region. Again, the updated ACT file may be corrected empirically or using the physical model described above. In some embodiments, when the difference between the measured temperature/tissue displacement and the target objective exceeds a threshold (e.g., 50%), the patient may be deemed unsuitable for the ultrasound procedure.

In various embodiments, prior to performing treatment on the patient, the ultrasound transducer 102 may be activated in accordance with the ACT file to the patient-specific printed 3D skull replica. By monitoring the treatment effects at the target location and/or on the non-target tissue (e.g., skull heating), patient suitability for ultrasound treatment may be predicted. Alternatively, the relative orientations and/or locations between the transducer elements 104 and the printed skull replica 302 may be adjusted until the desired treatment effects at the target location and/or safety to the non-target tissue is achieved. Again, acoustic signals at the target location in this new setup may be measured to update the ACT file.

Accordingly, the patient-specific 3D skull replica may be used to establish an ACT file that advantageously allows ultrasound parameters (e.g., locations, orientation, amplitudes and/or phase shifts) associated with individual transducer elements 104 to be corrected so as to compensate for beam aberrations caused by the skull; this approach thereby can create a high-quality focus at the target region and/or improve ultrasound beam shaping.

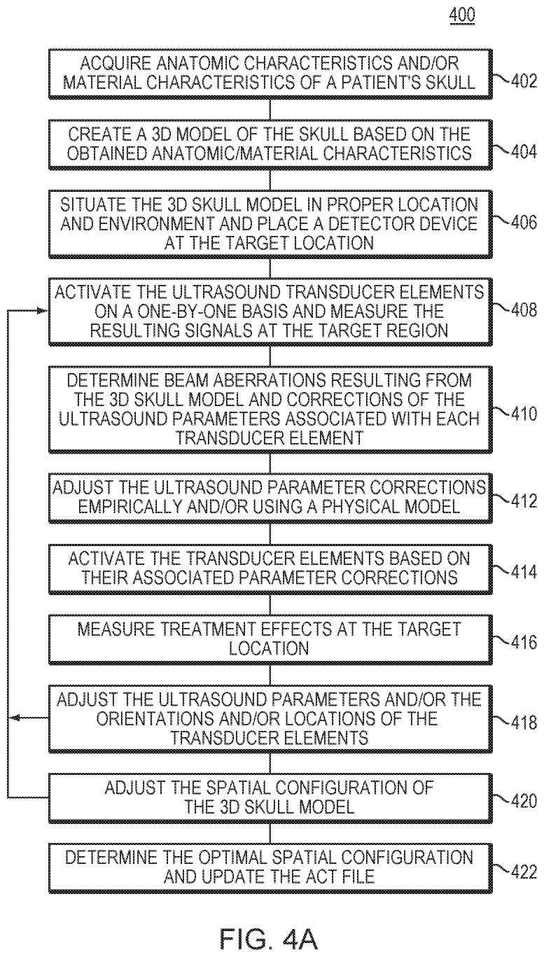

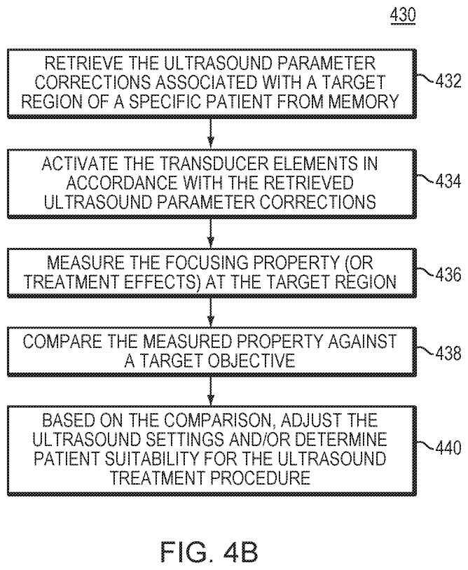

FIG. 4A is a flow chart illustrating an exemplary approach 400 for determining ultrasound parameter values for compensating for beam aberrations resulting from intervening tissue (e.g., the skull) located between the transducer and the target in accordance with various embodiments. In a first step 402, prior to the ultrasound procedure, anatomic characteristics and/or material characteristics of a patient's skull are acquired. In one embodiment, the anatomic/or material characteristics are extracted, manually or automatically using conventional tissue-analysis software, based on images acquired by the imager 112. In a second step 404, a 3D replica of the skull is created based on the obtained anatomic/or material characteristics thereof (e.g., using 3D printing). For example, the size and shape of the 3D skull replica may substantially match that of the patient's skull. In addition, the material made of the skull replica may be selected such that the speed of sound therein is substantially similar to that in the patient's skull. In a third step 406, the 3D skull replica is situated in the location and environment similar to that in which the patient's skull will be situated during the ultrasound procedure. Additionally, a detector device 304 may be placed at the target location. In a fourth step 408, the ultrasound transducer elements 104 are activated on a one-by-one basis to transmit acoustic signals to the target location and the detector device is activated to measure the resulting signals at the target region as they penetrate through the 3D skull replica. In a fifth step 410, the beam aberrations resulting from the 3D skull replica and corrections to the ultrasound parameters (e.g., amplitudes or phase shifts) associated with each transducer element 104 are determined based on the measured signals; the ultrasound parameter corrections may be stored in an ACT file in memory. Optionally, in a sixth step 412, the ACT file may be corrected empirically and/or using a physical model. In addition, the ultrasound transducer elements may be activated to transmit waves/pulses to the 3D skull replica in accordance with their associated parameter corrections in the ACT file (step 414). Subsequently, treatment effects at the target location and/or on the skull tissue may be measured using the imager and/or other suitable device (step 416). Based on the measured treatment effects, the ultrasound parameters and/or the orientations and/or locations of the transducer elements with respect to the 3D skull replica may be adjusted until desired target objective/safety are achieved (step 418). The ACT file may then be updated based on the adjusted element configurations. In some embodiments, the spatial configuration of the 3D skull replica and the transducer array is adjusted (step 420). Steps 408-418 are then iteratively performed to determine the ultrasound aberrations and ultrasound parameter corrections associated with each spatial configuration. Based on the determined aberrations, the spatial configuration corresponding to the optimal treatment effect can be determined, and subsequently, the ACT file may be updated based on the ultrasound parameter corrections associated with the determined spatial configuration (step 422).

FIG. 4B is a flow chart illustrating an exemplary approach 430 for optimizing focusing properties at the target during treatment in accordance with various embodiments. In the first step 432, the controller of the ultrasound transducer may access the memory to retrieve the ACT file associated with a target region of a specific patient. The transducer elements 104 may then be activated in accordance with the retrieved ACT file to commence ultrasound treatment and create a high-quality focus at the target and/or improve ultrasound beam shaping (step 434). Optionally, the focusing property at the target is directly or indirectly measured (e.g., using MR thermometry or MR-ARFI) (step 436). The measured property may then be compared against a target objective (step 438). In various embodiments, based on the comparison, the controller may adjust the ultrasound settings (e.g., by finely tuning the frequency, amplitudes and/or phase shifts of the transducer elements, deactivating a portion of the transducer elements 104 corresponding to large beam aberrations, etc.) to reduce beam aberrations, thereby improving focusing properties, or determine patient suitability for the ultrasound treatment procedure (step 440).

In general, functionality for determining ultrasound parameter values for optimizing focusing property at the target, including, for example, analyzing imaging data of the patient's skull acquired using an imager 112, determining anatomic/material characteristics of the skull based on the imaging data, causing a 3D skull replica to be generated using the anatomic/material characteristics, causing ultrasound beams to be applied to the target region through the 3D skull replica, measuring the acoustic signals at the target region, analyzing the measured acoustic signals to determine ultrasound parameter corrections for each transducer element, adjusting the determined ultrasound parameter corrections empirically or using a model, causing ultrasound beams to be applied to the target region based on the determined (or adjusted) ultrasound parameter corrections, monitoring the focusing property (or treatment effects) at the target region, adjusting the ultrasound parameter corrections and/or physical model based on the monitored value, adjusting the spatial configuration of the 3D skull replica with respect to the transducer and determining the optimal spatial configuration, as described above, whether integrated within a controller of the imager 112, and/or an ultrasound system 100, or provided by a separate external controller or other computational entity or entities, may be structured in one or more modules implemented in hardware, software, or a combination of both. The ultrasound controller 108 may include one or more modules implemented in hardware, software, or a combination of both. For embodiments in which the functions are provided as one or more software programs, the programs may be written in any of a number of high level languages such as PYTHON, FORTRAN, PASCAL, JAVA, C, C++, C#, BASIC, various scripting languages, and/or HTML. Additionally, the software can be implemented in an assembly language directed to the microprocessor resident on a target computer; for example, the software may be implemented in Intel 80×86 assembly language if it is configured to run on an IBM PC or PC clone. The software may be embodied on an article of manufacture including, but not limited to, a floppy disk, a jump drive, a hard disk, an optical disk, a magnetic tape, a PROM, an EPROM, EEPROM, field-programmable gate array, or CD-ROM. Embodiments using hardware circuitry may be implemented using, for example, one or more FPGA, CPLD or ASIC processors.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. A system for transmitting an acoustic beam comprising:
an ultrasound transducer comprising a plurality of transducer elements;
a three-dimensional (3D) printed tissue replica representing tissue intervening between the ultrasound transducer and a target anatomic region; and
a controller configured to:
(a) transmit a first ultrasound beam to the target region;
(b) measure the first ultrasound beam traversing the 3D tissue replica and arriving at the target region; and
(c) based at least in part on the measured first ultrasound beam, estimate a parameter value associated with at least one of the transducer elements for improving ultrasound beam shaping.

2. The system of claim 1, further comprising a detector device for measuring the first ultrasound waves at the target region.

3. The system of claim 1, wherein the estimated parameter value comprises at least one of a frequency, an amplitude, a time delay or a phase shift of the first ultrasound beam.

4. The system of claim 1, further comprising an imaging device for acquiring images of the intervening tissue, wherein the 3D tissue replica is generated based at least in part on the acquired images.

5. The system of claim 1, further comprising a 3D printer for generating the 3D tissue replica.

6. The system of claim 1, further comprising memory for storing the estimated parameter value associated with at least one said transducer element.

7. The system of claim 6, wherein the controller is further configured to retrieve the stored parameter value and cause at least one said transducer element to generate a second ultrasound beam based at least in part on the stored parameter value.

8. The system of claim 6, wherein the controller is further configured to:
sequentially cause at least some of the transducer elements to transmit ultrasound beams to the target region;
sequentially measure the transmitted ultrasound beams traversing the 3D tissue replica and arriving at the target region;
based at least in part on the measured ultrasound beams, estimate a plurality of parameter values associated with said some of the transducer elements; and
store the estimated parameter values in the memory.

9. The system of claim 1, wherein the controller is further configured to adjust the estimated parameter value using a physical model.

10. The system of claim 9, wherein the controller is further configured to use the physical model to predict a beam path from said at least one of the transducer elements to the target region based at least in part on a geometry of said at least one of the transducer elements and its location and orientation relative to the target region.

11. The system of claim 10, wherein the controller is further configured to:
use the physical model to predict the parameter value associated with said at least one of the transducer elements based at least in part on tissue characteristics of the intervening tissue along the beam path; and
adjust the estimated parameter value based at least in part on the prediction.

12. The system of claim 10, wherein the controller is further configured to:
use the physical model to predict the parameter value associate with said at least one of the transducer elements based at least in part on a material property of the 3D tissue replica; and
adjust the estimated parameter value based at least in part on the prediction.

13. The system of claim 1, wherein the controller is further configured to:
cause a second ultrasound beam to be transmitted to the target region;
measure the second ultrasound beam arriving at the target region after penetrating through the intervening tissue;
based at least in part on the measured second ultrasound beam, estimate a second parameter value associated with at least one said transducer element; and
adjust the estimated parameter value based at least in part on the estimated second parameter value.

14. The system of claim 1, wherein at least some of the transducer elements are activated to generate an ultrasound focus at the target region, the system further comprising a measurement system for monitoring treatment effects of the target region resulting from the ultrasound focus.

15. The system of claim 14, wherein the treatment effects comprise at least one of a temperature increase or a tissue displacement at the target region.

16. The system of claim 14, wherein the controller is further configured to adjust the estimated parameter value based at least in part on the monitored treatment effects.

17. The system of claim 14, wherein the controller is further configured to adjust a second parameter value associated with at least one said transducer element based at least in part on the monitored treatment effects, the second parameter value being different from the estimated parameter value.

18. The system of claim 17, wherein the second parameter value comprising at least one of a frequency, a location or an orientation.

19. The system of claim 1, wherein the 3D tissue replica and the ultrasound transducer have a spatial configuration, the controller being further configured to determine, based at least in part on the measured first ultrasound beam, an optimal spatial configuration of the 3D tissue replica and the ultrasound transducer.

20. The system of claim 19, wherein the spatial configuration comprises at least one of a relative orientation or location of the 3D tissue replica with respect to the ultrasound transducer.

21. The system of claim 19, wherein the controller is further configured to:
vary the spatial configuration of the 3D tissue replica and the ultrasound transducer;
repeat steps (a)-(c); and
based at least in part on the measured ultrasound beams, determine the optimal spatial configuration.

22. The system of claim 19, wherein the controller is further configured to determine the optimal spatial configuration using a physical model in addition to the measured first ultrasound beam.
